(19) United States
(12) Patent Application Publication
(10) Pub. No.: US 2004/0173211 A1
(43) Pub. Date: Sep. 9, 2004

(54) Powder inhaler

(75) Inventors: Heinrich Kladders, Muelheim (DE); Joerg Schiewe, Mainz (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim International GmbH, Ingelheim (DE)

(21) Appl. No.: 10/757,346

(22) Filed: Jan. 14, 2004

Related U.S. Application Data
(60) Provisional application No. 60/440,223, filed on Jan. 15, 2003.

(30) Foreign Application Priority Data
Jan. 14, 2003 (DE) 103 00 982

(51) Int. Cl. A61M 15/00

(52) U.S. Cl. 128/203.15; 128/203.21; 128/203.12

(57) ABSTRACT

The invention relates to powder inhalers in which at least part of the inner surface coming into contact with the powder aerosol is microstructured. Preferably, it is a powder inhaler operating on the Bernoulli principle (a Bernoulli inhaler).

### RELATED APPLICATIONS

Benefit of U.S. Provisional Application Serial No. 60/440,223, filed on Jan. 15, 2003 is hereby claimed, and which application is incorporated herein in its entirety.

### FIELD OF THE INVENTION

The invention relates to powder inhalers in which at least part of the inner surface coming into contact with the powder aerosol is microstructured. Preferably, it is a powder inhaler operating on the Bernoulli principle (a Bernoulli inhaler).

### PRIOR ART

A number of powder inhalers operating by various principles are known in the literature. As the Bernoulli inhalers are preferred according to the invention, these will be discussed first. What Bernoulli inhalers have in common is that the active substance to be delivered is stored in a cylindrical capsule and this capsule is inserted in a capsule chamber of the inhaler. The capsule chamber is usually cylindrical in shape, being somewhat longer and wider than the capsule so that the capsule is able to vibrate vertically (=axially), and also horizontally (=radially), but remains aligned substantially parallel to the chamber axis. The capsule chamber has an air inlet near one of its two ends and an air outlet opening in the region of the other end. The air outlet (air channel) leads to a mouthpiece. Within the scope of the present description of the invention, the direction running from the capsule chamber through the air channel to the mouthpiece defines the longitudinal axis and hence the axial direction. The direction perpendicular thereto defines the vertical or radial direction.

In order to deliver the active capsule contents, first of all the capsule is opened, normally at two places on its longitudinal casing. As a rule these openings are located close to the two longitudinal ends of the capsule. If an air stream is now generated from the air inlet towards the air outlet in the capsule chamber, it runs along the longitudinal axis of the capsule and has two effects: on the one hand the capsule is moved mainly along its longitudinal axis by the air stream. It can also vibrate to a small degree. On the other hand, the air flowing along the two capsule openings produces a lower pressure in front of the capsule openings than inside the capsule, so that the powder contained in the capsule is picked up by the air stream and thereby nebulised.

The capsules normally used for inhalers of this kind consist of two cup-like components which fit telescopically one inside the other. The outer shape of a composite capsule of this kind is that of a closed cylinder with hemispherical ends. The cylinder has a longitudinal axis and a transverse axis. The longitudinal axis is the axis which runs parallel to the generatrix of the cylinder casing. The longitudinal axis is longer than the transverse axis with the result that the longitudinal section of the capsule has an oval geometry and the cross section has a circular geometry.

Usually, the capsules for inhalable powders consist of hard gelatine but may also consist of a plastic material. In connection with this reference is made to EP 1100474.

DE 3345722 discloses an inhaler operating by the Bernoulli principle, consisting of two housing elements which are axially moveable towards each other, with a single capsule chamber. The inner surface of the hollow cylindrical capsule chamber is smooth.

WO 91/02558 discloses another Bernoulli inhaler wherein instead of a single capsule chamber there are a plurality of capsule chambers arranged in a similar manner to a revolver magazine. The open ends of this magazine are delimited by walls, the air inlet or air outlet being located only at one point in these walls. This magazine is mounted to be rotatable so that a capsule chamber is only connected to the air inlet, the air outlet and the cutting elements required to open the capsule in a certain position.

EP 0911047 also discloses a Bernoulli inhaler. This consists of a) a cup-shaped lower part open upwardly, b) a plate which covers the opening in the lower part and perpendicular to which is formed a capsule chamber of the kind described above, whilst on the capsule chamber is provided a button which is moveable against a spring which has two sharp spikes for opening the capsule, c) an upper part with a mouth tube which is connected to the capsule chamber, and capable of conveying a powder aerosol, and d) a lid. The elements a), b) c) and d) are joined together by a common hinge element so that they can be flipped relative to each other. In addition, this patent application describes a capsule holder wherein the capsule holder may be constructed as a hole in the plate b) and has ribs at the edge. The capsule is jammed into this capsule holder to hold it in readiness.

FR-A-2 146 202 describes a powder inhaler with a flat cylindrical chamber for accommodating a capsule. The capsule opened at the ends rotates during the inhaling process, driven by tangentially incoming air, about its transverse axis.

U.S. Pat. No. 4,069,819 describes a powder inhaler wherein the capsule is pierced through the base of the capsule chamber and during inhalation is set in motion by the air flowing in tangentially in the region of the base.

Other powder inhalers which do not operate by the Bernoulli principle include for example the inhalers disclosed in DE 3348370 and DE 3336486 which contain a disc-shaped blister pack comprising a plurality of blisters arranged in a circle. The individual blisters each contain a dose of a powdered medicament intended for inhalation. The blister pack contains the doses of the powdered medicament in non-encapsulated form. The blister pack is located in a chamber (storage chamber) in these inhalers and each of the blisters may be pierced at two opposite ends perpendicular to the plane of the disc. An air channel connects the opened blisters to the mouthpiece. The inhaler is described in more detail in DE 3336486 by way of example. This comprises a housing in which there is a chamber (storage chamber) which has an air inlet and in which there is a disc-shaped round blister with filled pouches of medicament. The blister is loosely attached to a round rotatable disc. Around the disc there are holes which are in axial contact with the pouches of medicament, i.e. the pouches and holes are located above and below one another. The chamber has an air outlet. The inhaler also comprises a piston which is arranged so that it can open a pouch of medicament by piercing it, so that the medicament is released into the chamber and can be breathed in through a mouthpiece. Reference is made to the drawings in the patent application and US patent specification.

DE 4106379 describes an inhaler into which a blister or the like for a powdered medicament can be placed. The blister consists of two strips of material which can be pulled away from each other, defining at least one container in which the medicament is located. The inhaler is provided with a device which pulls the two strips of material apart at an opening station in order to open a container. The user can inhale the powdered drug from the opened container through an outlet member, e.g. a mouthpiece connected to the opened container. One of the strips of material may also be a carrier strip with a plurality of pouches and the other strip of material may be a covering strip. Each pouch and the adjacent area of the covering strip then form a container. A drive device may be provided at the opening station for pulling the carrier strip and covering strip apart. This drive device may consist for example of two drive wheels (e.g. gear wheels) which hold the covering strip between them in driven engagement. In this case, too, each individual blister defines a kind of storage chamber in the inhaler, which is connected to the mouthpiece via an air channel.

U.S. Pat. No. 4,524,769 discloses a powder inhaler which comprises a nozzle (mouthpiece) for delivering the aerosol, an air channel and a storage chamber for the active substance. A membrane may be moved back and forth between the air channel and the storage chamber. This membrane has a plurality of means for receiving a metered quantity of active substance. Preferably, the membrane is a conveying membrane which as a device preferably comprises holes or perforated depressions for transporting active substance from the storage chamber into the air channel. In each case at least one of the devices between the air channel and the storage chamber is pushed back and forth. The device filled with a metered quantity of active substance is emptied in the air channel and then migrates back into the storage chamber to be filled there with another metered quantity of active substance by a filling means. The powder inhaler accordingly comprises means for moving the conveying membrane so that the filled devices containing the metered active substance formulation are transported from the storage chamber to the place of delivery of the active substance, the air channel, and from there the empty device is brought back to the storage chamber. A rota may also be provided in the nozzle.

Numerous inhalers comprise impact plates and the like mounted approximately in the region of the flow path, through which the nebulised active substance formulation is conveyed for delivery. The task of these impact means, if any, is to break up lumps.

In the development of any powder inhaler attention must be paid to ensuring that large accumulations of particles cannot be formed in the inhaler which would jeopardise its reuse on grounds of pharmaceutical quality. Therefore, and in order to ensure optimum delivery of the powder formulation, the inner surfaces of all the parts which may come into contact with the cloud of aerosol, particularly the storage chamber and the mouthpiece, are particularly smooth in construction.

To prevent contamination of a new aerosol cloud with old deposited particles it may be advantageous if the powder inhaler is cleaned after one or more inhalation processes, particularly those parts which come into contact with the powder formulation. This cleaning is necessary in order to minimise contamination of the next aerosol cloud to be breathed in, i.e. in order to ensure the pharmaceutical quality of the next aerosol cloud to be administered.

### DESCRIPTION OF THE INVENTION

Surprisingly, it has now been found that powder inhalers in which at least part of the inner surfaces which may come into contact with the powder aerosol are provided with a micro- or nano-structured surface, do not have worse delivery characteristics than powder inhalers with a smooth inner surface in these areas.

At the same time, surfaces of this kind are particularly suitable for washing simply with water, as the cleaning liquid simply rolls off the surface and carries any impurities with it.

No powder inhalers with a micro- or nano-structured surface are known from the prior art.

Therefore, it is an aim of the present invention to provide powder inhalers which can be cleaned more efficiently without affecting the delivery characteristics of the powder formulation.

A further aim is preferably to prepare Bernoulli inhalers of this kind, i.e. inhalers loaded with a capsule which contains the active substance formulation.

A further aim is to overcome the disadvantages known from the prior art.

### DETAILED DESCRIPTION OF THE INVENTION

The powder inhaler according to the invention essentially consists at least of a) a mouthpiece and b) an air channel opening into the mouthpiece which can be filled with the active substance formulation to be administered. Optionally, the powder inhaler contains a chamber, optionally equipped with an air inlet channel or an air opening, for receiving the active substance or the powdered, possibly compressed formulation containing the active substance, for receiving a capsule, a blister pack containing active substance and/or a conveyor belt containing active substance, each of which contains the formulation holding the active substance. Means for opening the blisters or capsules are optionally provided. In the storage chamber or air channel the pharmaceutical composition is mixed with air and conveyed to the user's mouth through the mouthpiece.

In the case of Bernoulli inhalers, in particular, an air channel may connect the chamber to the mouthpiece. In this case the chamber may also comprise another air inlet channel or an opening. The chamber in the Bernoulli inhalers is preferably a chamber for holding a capsule (capsule chamber) which is provided according to the invention with means for opening the capsule at the side. The capsule chamber is constructed so that the reservoir capsule inserted can essentially only perform a movement in the longitudinal direction when an air current travelling essentially parallel to the longitudinal axis of the capsule passes through the capsule chamber and has only limited play along the transverse axis thereof. Such capsule chambers are typical of Bernoulli inhalers. Within the scope of the present invention the chamber may also be designated a storage chamber or metering chamber.

The powder inhalers known from the prior art have a smooth, unstructured inner surface. The same applies to other elements which may come into contact with the powder aerosol. According to the invention the structure of the inner surface of these parts having a critical inner surface is different from an unstructured or smooth surface. By unstructured or smooth is meant, for the purposes of the present invention, surfaces which do not have a surface structure as described within the scope of the invention.

For the powder inhalers according to the invention any of the inhalers described in the “prior art” section hereinbefore may be used. To avoid repetition, the features of these inhalers mentioned above will not be discussed again at this point but reference is made specifically to this section. The inhalers according to DE3345722, WO91/02558 or EP0911047 are of particular interest.

The devices known by the brand names “TURBOHALER®”, “EASYHALER®”, “DISCUS®” and “HANDIHALER®” may be mentioned by name, in particular.

The features mentioned in the prior art section hereinbefore for the powder inhalers generally described also apply to the powder inhaler according to the invention, with the exception of the parts which come into contact with the powder aerosol in relation to the configuration of the inner surface, and will therefore not be mentioned again at this point. In a preferred inhaler according to the invention the capsules mentioned in the same section may be used.

Within the scope of the present invention the inner surfaces of the parts which come into contact with the powder aerosol, i.e. the surfaces which are most in contact with the powder aerosol, are referred to as the critical surface. The critical surfaces specifically include the mouthpiece and the air channel opening into the mouthpiece. The inner surface of the lower part or the impact plate of an inhaler according to EP0911047 described hereinbefore may optionally also be provided with the structure according to the invention.

In the case of Bernoulli inhalers (c.f. for example DE3345722, WO91/02558 or EP0911047), which are preferred according to the invention, the inner surface of the capsule chamber are also part of the critical inner surface.

In the case of inhalers according to DE3348370, DE3336486 and DE4106379 the chamber for accommodating the blister pack may also be counted as a critical surface.

In the case of an inhaler which uses the principle described for U.S. Pat. No. 4,524,769 hereinbefore, the storage chamber may also have a critical surface.

In inhalers with a separator this may also be a critical surface. A separator of this kind is described for example in EP0633792.

According to the invention at least some of the critical surface of the powder inhalers is provided with a micro- or nano-structure. Surfaces with a micro-structure having self-cleaning properties are described in EP772514 or DE20114878U1, to which reference is hereby made.

Preferably, at least 20% of the inner surface of the mouthpiece is micro- or nano-structured, more preferably 50% and most preferably at least 75%.

In the case of Bernoulli inhalers, preferably also or only the inner surface of the capsule chamber is micro- or nano-structured over at least 20% of its surface, more preferably at least 50% and most preferably at least 75%.

In the case of inhalers with blister packs or conveyor belts the inner surface of the air channel opening into the mouthpiece is micro- or nano-structured over at least 20% of its surface, more preferably at least 50% and most preferably at least 75%.

The outer shape of the parts which have a critical inner surface is of no significance for the purposes of the present invention and may resemble the configuration of the devices known from the prior art. Thus, in Bernoulli inhalers, for example, the outer shape of the capsule chamber is determined by its position and any movements thereof in the inhaler or the movements of other parts of the inhaler around the capsule chamber.

The structuring of the critical surface according to the invention is achieved by providing raised portions and depressions at least on areas of the critical inner surface. This produces the structural shapes according to the invention.

The raised portions and depressions may be in the form of peaks, spheres, flat surfaces, wedge shapes, hemispherical shapes, etc.

They may be randomly arranged or ordered, e.g. in rows, circles, in a zigzag, meandering, etc.

The spacing between the raised portions on the surface structure is in the range from 0.1 to 200 microns, preferably 0.1 to 100 microns. Distances of 0.1 to 1 micron are more preferred. The spacings between the raised portions may differ from one another.

To ensure optimum delivery of the particles, spacings of 1 to 25 microns should be avoided.

Accordingly, the preferred dimensions of the structure of the structural shapes are less than the diameter of the aerosol particles, which are typically 1 to 20 microns, preferably 1 to 5 microns. The height of the raised portions or the depth of the depressions are in the range from 0.1 to 100 microns, preferably 0.1 to 50 microns. Spacings of 0.1 to 10 microns are most preferred.

Preferably the raised portions of the surface structures are close enough together to ensure that hydrophilic drops of liquid, e.g. drops of water, roll of the raised portions without actually touching the underlying area. At the same time the raised portions of the surface structures should not be too close together or the depressions should not be too flat so as not to form a sealed surface, with respect to the droplet size of the liquid, in which the surface forces between the drops and the surface come into effect fully. It is desirable that the height of the raised portions from the base should increase as the distance between the raised portions increases. Preferably, the surfaces have raised portions measuring 0.1 to 50 microns wherein the spacing between the raised portions is 0.1 to 100 microns.

The micro-structured surfaces preferably have at least two different kinds of structural shapes, the raised portions and/or depressions of which are distinguished from one another by different shapes, heights and/or intervals. Individual examples of the two different structural shapes may be at different spacings from their neighbours. Details may be found in the prior art.

Preferably, the critical surfaces consist of hydrophobic materials or materials which have been given a durable hydrophobic finish or they are coated with such materials and the raised portions cannot be detached by water or water-containing detergents. The materials used may be plastics, metals, ceramics, glass, etc.

Preferred materials are glass and/or ceramics and/metals and/or plastics such as polyethylene, polypropylene, polycarbonate, polyacrylates, polyesters, silanes, etc.

Plastics are preferred. If desired, a plastic of this kind may be provided with a coating of another plastic which carries the surface structure.

Structured surfaces of this kind may either be produced either by forming the surface structures during the manufacture from hydrophobic materials or by subsequently subtracting or adding material to the surfaces. These processes include subsequent stamping, etching, laser ablation, galvanic machining, adhesive bonding of a structured film, adhesive bonding of a powder, spraying with suspensions, depositing sublimates.

Finally, it is possible to create self-cleaning surfaces of this kind on objects by subsequent provision of a durable hydrophobic surface on previously produced surfaces with the desired structures.

One possible way of subsequently making a surface durably hydrophobic is by subsequently silanising surfaces with the desired structures which have been prepared beforehand. Silanising may be carried out on any materials which are naturally hydrophilic but capable of reacting with the reactive groups of the silanes so that finally the surface consists of the hydrophobic groups of the silanes.

In order to produce the desired surface structures during the actual manufacture from hydrophobic polymers the objects may be produced in moulds which contain the negative of the desired surface structure.

It is also possible to apply the hydrophobic polymers in the form of solutions and/or dispersions which produce the desired surface structures when dried and cured.

Such structures are formed for example from self-organising polymers or under conditions as known in principle from the manufacture of matt paint surfaces.

If it is not possible or not desirable to create the desired surface structures from the outset, this may also be done subsequently, e.g. by subsequent stamping or etching. Stamping may be carried out, for example, using heated or heatable stamps. The etching may be carried out using the known means for chemical etching or by physical methods such as ion etching with oxygen or other irradiation which leads to roughening of the surface and a surface structure which can be used according to the invention.

The method in which a surface structure is produced depends on the material used and the desired micro-structure.

It has also been found that it is also possible to obtain the desired surface structure by adhesively bonding a powder of the hydrophobic polymers. Powders of hydrophobic polymers with the desired particle size can be obtained. Optimum results are only achieved, however, if powders with a relatively narrow particle size distribution are used.

As already described, the inner surface of the mouthpiece in all kinds of powder inhalers is one of the critical surfaces. The shape of the mouthpiece is essentially defined by its function.

The mouthpiece, which is generally tubular and optionally somewhat flattened may be arranged axially or at an angle to the axis of the air channel connected to it or offset to one side of this axis.

In the case of the preferred Bernoulli inhalers, apart from the inner surface of the mouthpiece the inner surface of the capsule chamber is one of the critical surfaces. Therefore, these two elements will be discussed in more detail at this point. In the simplest case the mouthpiece is a tube which is connected to the capsule chamber at one end and is open at the other end.

It may be constructed in the form of a cap which is fitted on to a lower part of the inhaler which contains the capsule chamber. This cap may be hinged to the edge of the inhaler housing so as to be pivotable about an axis extending perpendicularly to the longitudinal axis of the inhaler. The mouthpiece and the lower part of the inhaler housing may, however, also be fixed to one another by a conventional push-fit connection. In any case, access generally, to the capsule chamber and to the cutting device in the lower housing part, on the one hand, and to the inner components such as the perforated plate and the upper housing part (of the mouthpiece-cap) is made substantially easier by the removability or pivotability of the two components.

In order to replace used capsules with fresh ones, in an embodiment of this kind the mouthpiece is flipped upwards or the push-fit connection between the mouthpiece and the lower housing part is undone. The capsule chamber is then freely accessible, so that the emptied capsule can be removed and a full one inserted. The device is then flipped shut or pushed shut.

The inner shape of the capsule chamber is typically such that it comprises a cavity open on two sides for accommodating a disposable capsule for pharmaceutically active inhalable substances. Preferably, these two openings are provided at opposite ends or immediately adjacent these ends. The inner form may for example be a preferably uniform cylinder or cuboid. Preferably the inner configuration resembles a cylinder.

The dimensions of the capsule chamber are matched to those of the capsule. The cavity preferably has a diameter which is 1.1 to 2.5 times as great as the capsule diameter. Preferably, the cross section is 1.1 to 2.2 times, particularly 1.2 to 1.6 times as great as the capsule diameter.

The length of the inner cavity of the capsule chamber is 1.02 to 2 times as great as the length of the capsule, preferably 1.04 to 1.8, particularly 1.1 to 1.6 times as great as the length of the capsule. The diameter of the chamber should be less than the length of the capsule, so that the capsule is held in the longitudinal direction in the chamber and cannot tilt to one side.

As an illustration some examples of typical capsule dimensions will now be given, indicating the size of the capsule chamber.

Total length of the closed capsule: 26.1±0.3 mm; 23.3±0.3 mm; 24.2±0.3 mm; 21.7±0.3 mm; 19.4±0.3 mm; 18.0±0.3 mm; 15.9±0.3 mm; 14.3±0.3 mm; 11.1±0.3 mm.

Outer diameter of the capsule body: 9.55 mm; 8.18 mm; 7.36 mm; 7.34 mm; 6.63 mm; 6.07 mm; 5.57 mm; 5.05 mm; 4.68 mm.

External diameter of the capsule caps: 9.91 mm; 8.53 mm; 7.66 mm; 7.64 mm; 6.91 mm; 6.35 mm; 5.83 mm; 5.32 mm; 4.91 mm.

The standard commercial capsules are size 3, which is known at least in Germany. In the telescopic capsules described the diameter of the upper part is 5.83 and the diameter of the lower part is 5.57 mm.

The capsule chamber has two openings, an inlet for incoming air and an air outlet. The air inlet is smaller in cross section than the capsule chamber so that in this region of the capsule chamber the flow velocity of the air is relatively high and a powder in the capsule is delivered by the Bernoulli effect. The air inlet opening is conveniently arranged centrally in the base of the chamber. On the air outlet side there may be a perforated plate or other device such as projecting components to prevent a capsule moving in the capsule chamber from blocking the air outlet or any capsule fragments formed from being sucked into the mouthpiece. The perforated plate may for example be part of a funnel-shaped connecting member which can be fitted on to the start of the inhalation channel leading to the mouthpiece in such a way that the edge of the funnel with the perforated plate engages in a plate-shaped insert which forms the base of the mouthpiece. The perforated plate may, however, also be replaceably fixed by jamming it between the funnel edge of the connecting member and a stop of the plate-shaped insert.

A plurality of openings may also be provided as the outlet opening. The cross section available for the air to flow out of the capsule chamber is conveniently greater at every point than the air inlet opening so that the air charged with the pharmaceutical composition can flow out unimpeded as far as possible. The air outlet opening is expediently arranged centrally in the top of the chamber but may also be arranged to one side in the top region.

The provision of the two openings is intended to guide an air stream axially through the capsule chamber.

The capsule chamber has at at least one point along its longitudinal axis (in relation to the interior of the capsule chamber) an opening for or a connection to a cutting device which is provided with at least two sharp spikes or cutters for piercing or cutting open a capsule located in the capsule chamber. The cutting device is moveable into the interior of the chamber counter to the pressure of the spring and is operated by means of a spring mounted actuating button. As the height of the capsule chamber is determined by the length of the pharmaceutical capsules, the points or cutters of the cutting device are preferably located close to the top or bottom end of the capsule chamber. The side wall of the capsule chamber may have radial bores or oblong slots in the region of its top and bottom end which face the spikes or cutting edges and serve to allow the spikes or cutters to pass through. The dimensions of these bores/slots are matched to the cross section of the spikes or cutting edges.

In a preferred embodiment of the Bernoulli inhaler according to the invention the guide for the spikes of the cutting device comprises a sealing plate. In this way the seal between the capsule chamber in the inhaling position and the cutting device is improved. For the spring mounting of the sealing plate it is possible to use the spring which resets the actuating button for the cutting device.

Finally, in another embodiment, a lever system is provided for actuating the cutting device. This lever system is preferably actuated by an actuating button mounted on the base or side of the housing of the inhaler. The lever system may consist of a rocker and a toggle lever, while the actuating button acts on one end of the rocker and the other end of the rocker presses on one end of the toggle lever, the other end of the toggle lever secured to the cutting device pushing the cutting device forward. The rocker and toggle lever are preferably mounted to be pivotable about axes in holders secured to the housing.

The capsule is supposed to be opened close to both its ends for the inhalation process. The hemispherical caps of the capsule should not be damaged thereby. This is important because the capsule or caps of the capsule act as a sort of valve. Because of the pressure conditions the capsule is pulled towards the inlet opening counter to the inflowing air and closes it off. As the user continues to suck on the mouthpiece, suction is produced in the capsule chamber by which the capsule is pulled towards the air outlet with the inflowing air. The suction now formed at the air inlet ensures that the capsule is pulled towards the inlet opening again. The entire process is repeated in rapid succession as long as the patient continues to inhale through the mouthpiece and sets the capsule vibrating strongly in the axial direction.

Preferred Bernoulli inhalers are those described hereinbefore as embodiments of DE 3345722, WO 91/02558 or EP 0911047. Reference is hereby made once again to the features mentioned in this section. The inhaler as described hereinbefore in connection with EP 0911047 is particularly preferred.

In inhalers of this kind there can only be one capsule chamber according to the invention, in accordance with the remarks on DE 3345722 or EP 0911047. However, the capsule chamber may also be part of a capsule chamber magazine as described in WO 91/02558.

An inhaler of this kind has a revolver magazine with a plurality of usually tubular chambers each loaded with one capsule. The magazine is covered at each of its two open ends by a plate, one plate containing the air inlet opening and axially thereto the other plate containing the air outlet opening. As the magazine is rotatably mounted within these plates, one of the chambers can be pivoted into place between the two openings and thus form part of the continuous channel for the inhaled air. After an inhalation process has ended the revolver magazine is further rotated until the next chamber enters the air throughflow channel. One of the two plates may be separated from the magazine, for example, in order to remove used capsules from the chambers, or else the entire magazine can be removed for refilling, for example.

According to this feature of the invention the revolver magazine is releasably mounted in the inhaler housing. After the capsules in the revolver magazine have been used the entire revolver magazine can be replaced or refilled with capsules.

The inhaler housing may have an eccentrically mounted pin on to which the revolver magazine can be fitted.

In order to fix the position of the revolver magazine it may be provided with recesses associated with the capsule chambers for a spring-mounted locking bolt arranged in the inhaler housing. The recesses are arranged so that the locking bolt only engages therein when one of the capsule chambers is located precisely between the air inlet and outlet.

In this way it is possible to ensure that the revolver magazine does not move during the inhalation. The spring mounting of the locking bolt should be selected with regard to the spring constant so that accidental rotation of the revolver magazine is prevented by the locking but on the other hand if greater force is applied the revolver magazine can be rotated out of its locked position. Conical shapes for the free end of the locking bolt and suitably shaped recesses have a supporting effect.

The locking bolt is preferably arranged coaxially with the air throughflow channel underneath the capsule chamber and has a through-bore which simultaneously forms the air inlet in the base. Preferably, the locking bolt is centrally mounted in the inhaler housing. According to another embodiment of the invention the locking bolt is acted upon by a spring the other end of which abuts on a stopper releasably fixed in the inhaler housing, which also has a central through-bore which is part of the air throughflow channel.

In a preferred embodiment the recesses for engagement of the locking bolt in the base are arranged in the base plate of the magazine, concentrically with the air inlet bores of the capsule chambers and like the casing constructed in the form of a flat truncated cone with its base facing outwards. Thus, these recesses are conical or funnel-shaped widenings of the air inlet bores, the widened area facing the locking bolt. The slopes produced by the widening correspond approximately to the chamfers on the top of the locking bolt.

In a preferred embodiment these recesses have an encircling stop edge on the base of the casing of the truncated cone, but also in the base plate, which acts as a rotation preventer or stop for the head of the locking bolt when the latter has engaged in the corresponding recess. Because of this stop edge the magazine cannot be turned any further once the locking bolt has engaged.

According to another feature of this embodiment the said stop edge takes up only part or half of the periphery of the conical recess, i.e. the funnel-shaped widening, and is arranged so that when the locking bolt is engaged it prevents rotation of the magazine in one direction but allows it in the other direction, as the sloping wall of the funnel-shaped widening merges smoothly into the exterior of the base plate.

In another preferred embodiment only one of the recesses has a stop edge which takes up the entire circumference of the recess so that when the locking pin is engaged it is impossible for the magazine to rotate in this recess. This position is then regarded as the end position of a magazine in which all the capsules have been used. In this embodiment, all the other recesses only have a rotation preventer on one side, i.e. effective in one direction, so that the magazine can only ever be rotated in the direction in which a capsule chamber containing an unused capsule is brought into play, until the end position described above in which locking is complete is reached. The user then knows that the magazine has to be loaded with fresh capsules once this last capsule has been used.

In another preferred embodiment a tongue may be fixed to the locking bolt which extends as far as a stop on the inside of the operating button of the cutting device when the locking bolt assumes its upper stop position with the revolver magazine removed. In this position the said tongue acts as a barrier for the cutting device. When the magazine is inserted the locking bolt is pressed down again and in this way the barrier for the cutting device is removed.

The actuation of the cutting device may also be coupled to the rotary movement of the capsule magazine, so that at the press of a button first a capsule chamber is brought into the correct position and then the cutting device is engaged.

If the revolver magazine and the part of the inhaler housing adjacent thereto are constructed with n angles, where n is a whole number indicating the number of capsule chambers, the side surfaces of the inhaler housing part and of the revolver magazine may advantageously be aligned when the magazine is in the correct position. It is then possible to determine immediately from outside whether the chamber is located in the air channel defined by the air inlet and the air outlet.

In addition to the inhalers mentioned, the invention may also be used in inhalers as disclosed by DE 3336486 (U.S. Pat. No. 4,627,432, U.S. Pat. No. 4,778,054), DE 3348370 (U.S. Pat. No. 4,627,432, U.S. Pat. No. 4,778,054), DE 4106379 (U.S. Pat. No. 5,590,645, U.S. Pat. No. 5,860,419, U.S. Pat. No. 5,873,360, U.S. Pat. No. 6,032,666, U.S. Pat. No. 20,020,053,344, U.S. Pat. No. 20,020,066,451, U.S. Pat. No. 6,378,519) or for example DE 3274065 (U.S. Pat. No. 4,524,769).

Characteristic features thereof may be found in the section on prior art.

The inhaler according to the invention makes it possible to deliver the pharmaceutical composition more reliably than with devices known from the prior art, with lower standard deviations, and ensures good cleaning thereof.

It could not have been foreseen that the structured surface structures with raised portions and depressions in a powder inhaler would not have a detrimental effect on the characteristics of delivering a powdered formulation as an aerosol.

In particular, there was a fear that solid particles would adhere more strongly to a structured surface than to a smooth one and hence would lead to poorer delivery of the powder inhalers and/or contamination of the surface.

However, it has been found that the powder particles suitable for administration by inhalation do not adhere any more strongly to a microstructured surface than to a smooth surface and any particles adhering can be removed without trace using water. Drops of water landing on it force their way through the microstructures and pick up any particles lodged therein.

Instead of or in addition to the remarks relating to powder inhalers, the inside or outside of the supply capsules may also be provided with the microstructured surface according to the invention. A capsule of this kind is preferably cylindrical with tapering ends. It consists of at least two partial elements fitting telescopically into one another. Preferably, these capsules have a longitudinal axis and a shorter transverse axis. The longitudinal axis is the one running parallel to the generatrices of the cylindrical casing. The longitudinal axis is longer than the transverse axis, so that the longitudinal section of the capsule is oval while the cross section is circular in shape.

Preferably, the minimum of two partial elements are pushed into one another in the direction of the longitudinal axis.

Details of the capsule construction can be found in the section on prior art and elsewhere in this description. In particular, details can be found in EP 1100474, to which reference is hereby expressly made.

Analogously thereto, the blister packs (DE 3348370, DE 3336486, DE 4106379) or conveyor belts (U.S. Pat. No. 4,524,769) mentioned for the inhalers described above may also be coated on their inside and outside with microstructured surfaces of this kind.

Such blisters may have a blister bed with well-like depressions or cups sealed off by an overlying film.

In these blisters, the cups may be arranged side by side as in a string of pearls or may be arranged in rows. The blister beds may consist of a plastic or aluminium foil. The same is also true of the sealing films.

Materials which may be used are those disclosed in the prior art, such as plastics, aluminium foils, etc.

The invention will be described in more detail hereinafter by means of Examples and Figures.

### EXAMPLES

### Example 1

A smooth surface of plastics such as Resopal or polyethylene is provided with a thin even coating of an adhesive such as UHU PLUSs and then coated with a Teflon powder such as Hosta flons TF 9205 (average particle size 7 microns). After curing a surface is obtained from which deposited particles such as soot and paint powder can be rinsed off with water.

### Example 2

A smooth hydrophobic material such as PTFE is heated until it is plastically deformable. Then a high mesh screen from offset printing is pressed onto the surface and removed again.

After cooling, a surface is obtained with a regular arrangement of elevations and depressions of similar height.

By using different screens of different mesh size and thickness, the dimensions can be altered and adjusted to an optimum. The properties of the surfaces thus obtained are optimum when the elevations have rounded tips. These surface structures may, of course, also be produced by heated stamping tools or rollers. Corresponding films may be adhered to a different smooth substrate.

### Example 3

The method of adhering fenoterol to polyester films with a structured acrylic layer is investigated.

Film 1 bears structures in the region of 0.5 microns.

Film 2 bears structures in the region of 2 microns.

Film 3 bears structures in the region of 2 microns, with a 10 micron superstructure.

A polyester film with an unstructured acrylic layer is used as reference.

The films of Example 3 were each stuck into the lid of a container. Defined amounts of powdered fenoterol were applied to the film sections using a cascade impactor. The impactor was operated at a flow rate of 39 l/min and the film sections were placed at precipitation stage 2. This test set-up makes it possible to deposit on the films defined amounts of powder with particles having an aerodynamic diameter of about 4.3 to 5.2 microns.

The heaps of powder do not form a monolayer but rather an agglomerate of particles of powder. The adhesive forces between them differ from those between the powder and the film. The lid is placed on a container and put in a centrifuge. By running it at various speeds the powder is detached from the film by centrifugal force and spun into a collecting container. Then the difference between the mass of powder in the collecting container and on the film section in the lid is determined.

Results:

If the percentage of powder detached is plotted as a function of the ratio G of centrifugal force to the weight force of the particles this function is shown as a sigmoid curve which rises sharply at first, becoming convergent at higher G levels. The G value is also a measurement of the adhesion of a powder particle to the surface of the film.

It is found that-the curves plotted so not differ significantly for any of the films and no difference can be observed from the unstructured control material. What is more or less common to all the curves is that the powder only begins to be detached from the films upwards of G values of 200 to 800. At G values of 57000 the percentage quantity of powder detached is from 47 to just 60%.

For

Film 1 the value G=57000 is between about 49% and 56%,

Film 2: the value G=57000 is between about 46% and 57%,

Film 3: the value G=57000 is about 60%.

unstructured reference film: the value G=57000 is about 47 to just 60%

The curves measured lead one to conclude that on structured surfaces the characteristic structural size of which is less than the diameter of the particles, the forces required to detach the particles from the surface are no greater than on an unstructured surface, i.e. no higher adhesive forces occur which would lead to heavier deposits.

### FIGURES

FIG. 1 shows a typical two-part capsule which may be provided with the microstructured surface according to the invention.

FIG. 2 shows an inhaler in which the capsule according to the invention can be used.

FIGS. 3a to d show a powder inhaler with a revolver magazine in which the capsule according to the invention can be used.

FIG. 4 shows a powder inhaler with an upper and lower part movable relative to each other.

FIGS. 5 to 9 show examples of surface structures.

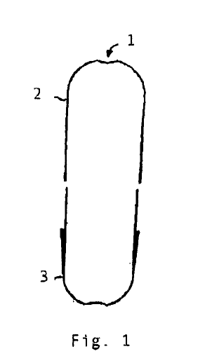

FIG. 1 shows a capsule (1) known from the prior art, consisting of a capsule cap (2) and a capsule body (3). It is apparent that the outer diameter of the capsule body is smaller than that of the capsule cap over wide areas. This is particularly noticeable in the region of the hemispherical end of the capsule body at the bottom.

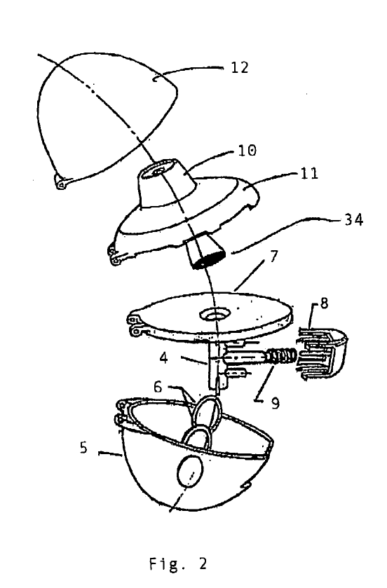

FIG. 2 shows how an inhaler may be constructed in which a capsule chamber according to the invention is integrated. Located in a lower part (5) optionally with two windows (6) is a plate (7) connected to the capsule chamber (4). The capsules in the capsule chamber (4) are opened by means of a button (8) provided with two specially sharpened spikes which is pressed in counter to the pressure of the spring (9) and thereby cuts open or pierces the capsule in the chamber in two places. As the user inhales through the device using the mouthpiece (10) which is connected to the upper part (11), the air enters the lower part (5) and from there goes into the capsule chamber (4) at the lower end. The device is closed off by a lid (12), which is hinged to the lower part (5), the plate (7) and the upper part (11), so that when the lid is closed dust cannot enter the device. In the plate (7) there may optionally be capsule holders in the form of blind bores. Advantageously, there is a perforated plate (34), which is fixed to the lower end of the mouth tube (10) or of the inhalation channel leading to the opening of the mouthpiece and, when the inhaler is in the closed position, covers the air outlet opening of the capsule chamber (4). The drawings do not show optional snap-fit hooks on the side of the mouth tube (10) or of the upper part (11) which is oriented towards the plate (7), which are capable of engaging in the plate (7). In this case the plate (7) has suitably complementary devices (depressions or holes). Projections or snap fit hooks may also be provided laterally on the plate (7), for example to enable the plate (7) to engage in the lower part (5). The above mentioned devices for engaging the mouthpiece (10) or upper part (11) in the plate (7) or the plate (7) in the lower part (5) are such that the individual elements can easily be separated from one another again. In addition, a lug may be formed on the point on the lid (12) which is located above the button (8) in the closed position so that this lug engages in a depression on the top of the button (8) and blocks the button (8), so that the button (8) cannot be pressed in the closed position. This prevents the capsule from being accidentally perforated prematurely once it has been inserted in the capsule chamber.

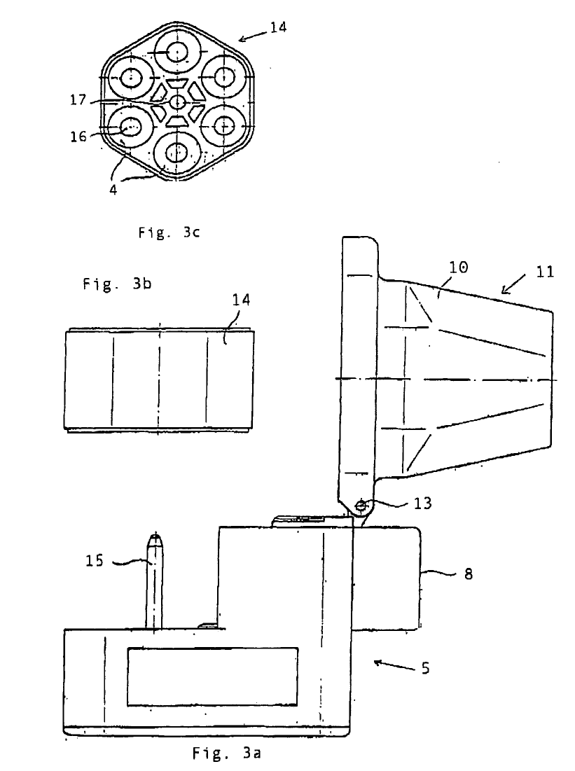

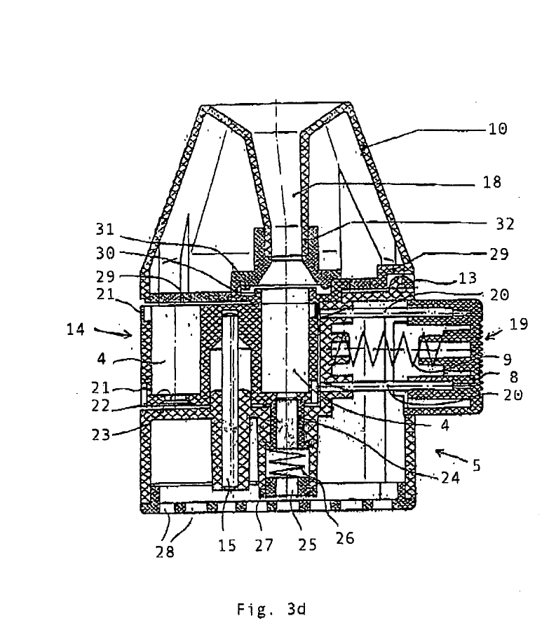

FIG. 3: As can be seen from FIGS. 3a, 3b and 3c, an inhaler with a revolver magazine consists essentially of an inhaler housing (5) with a mouthpiece (10) which is hinged laterally to the upper edge of the inhaler housing (11) so as to be pivotable about an axis (13), and a revolver magazine (14) with the capsule chambers (4) for accommodating the capsules. The revolver magazine (14) can be fitted on to a pin (15) eccentrically mounted in the inhaler housing (5). After the revolver magazine (14) has been pushed on the mouthpiece (10) is moved into its normal position—as a cap on the housing; the inhaler is ready for use. A capsule (not shown) can now be perforated by pressing the button (8). As can be seen from FIG. 3c, the revolver magazine (14) in this case has 6 chambers (4) for accommodating the capsules (not shown). The base of each chamber (4) has an air inlet bore (16). In addition, the revolver magazine (14) has an axial guide (17) for the pin (15).

As may be seen from FIG. 3d, the inhaler has, adjacent to the chamber (4) mounted underneath the inhalation channel (18), the cutting device (19) which is operated by means of the button (8). This cutting device (19) has two spikes (20) which can be radially inserted into the upper and lower part, respectively, of said chamber (4), the outer wall of the revolver magazine having weakened or frangible regions (21) at suitable points to assist the insertion of the spikes (20). The spikes (20) serve to open the capsule located in the chamber (4) close to the upper and lower ends thereof. The revolver magazine (14) also has, underneath the bores (22), conical recesses (23) in which a locking bolt (24) can engage as soon as the corresponding chamber (4) is coaxial with the air inlet or inhalation channel (18) of the inhaler housing. The locking bolt is also conically formed at its end engaging in the recess (23). At the opposite end it is acted upon by a spring (26) which bears on a stopper (27) releasably fixed in the inhaler housing. This stopper, like the locking bolt, has a central through-bore which acts as an air inlet (25).

In order to prepare the inhaler, with the revolver magazine (14) in place, this magazine is rotated so that one of the chambers (4) is brought into a position in which the bore (22) in the base or the conical recess (23) is aligned coaxially with the air inlet opening (25). The positioning of the chamber (4) is made easier by the engagement of the locking bolt (24) in the recess (23). After the bolt has engaged, the air inlet opening (25) and the base opening (22) in the chamber (4) are in alignment. The cap of the capsule is positioned on the base opening (22) and closes it off. By actuation of the button (18) counter to the force of a spring (9) the cutting edges (20) are moved radially towards the chamber (4), first piercing the weakened regions (21) or entering corresponding openings in the side wall of the revolver magazine and finally opening the capsule at the top and bottom close to its ends. The tapering caps of the capsules should not be destroyed as they are intended to act as a kind of valve.

When air is then sucked through the mouthpiece (10), the air flowing into the chamber (4) from the base openings (28) in the housing (5) and the air inlet (25) sets the capsule vibrating violently, produces turbulence in the powder in the capsule, mixes with it and is finally inhaled. The mouthpiece (10) is generally tubular in construction but may also be adapted to the shape of the mouth and flattened. Similarly, as an alternative to the embodiment shown, the mouthpiece may be arranged axially or at an angle to the axis of the chamber or laterally offset from the axis of the chamber.

At the base, the mouthpiece (10) may be provided with a plate-shaped insert (29) which is essentially solid. This plate-shaped insert (29) may also have perforations, however. Moreover, the start of the inhalation channel (18) may be covered with a screen which prevents the capsule or capsule fragments from being inhaled into the inhalation channel (18) in the mouthpiece. Alternatively, projections may be provided on the wall at this point to hold the capsule back. The perforated plate is then preferably arranged in the centre of the plate-shaped insert (29), advantageously clamped between a stop (30) on the plate (29) surrounding the air throughflow and the edge of a funnel-shaped connecting member (31), which is fitted on to the beginning (32) of the inhalation channel (19) in such a way that the edge of the funnel faces the plate-shaped insert (29) and engages therewith. The alternatively provided projections may also be arranged at this point.

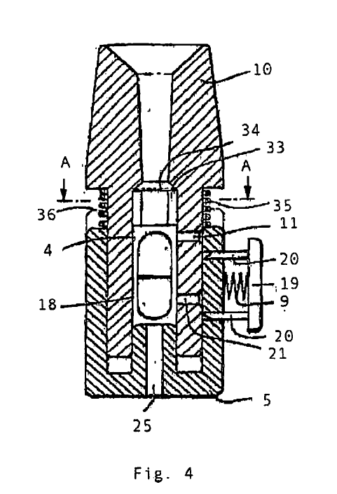

The embodiment of the inhaler according to the invention as shown in FIG. 4 consists of the lower part (5) and the mouthpiece (10), which are fitted together. The lower part contains the air inlet channel (25) which is connected to the air inlet into the capsule chamber (4). The cutting device (19) is held in its normal position by a spring element (9). The mouthpiece (10) contains the capsule chamber (4). Projections (33) which limit the play of the capsule project into the extension of the capsule chamber. A perforated plate (34) prevents fragments of capsule from being inhaled, for example. The inhaler may be axially compressed counter to the pressure of a spring element (35), the upper edge of the lower part reaching the position (36). In this position the blades or points (20) of the cutting device (19) may penetrate through the opening (21) into the capsule chamber (4) and open the capsule secured therein.

In order to use the inhaler according to FIG. 4 the lower part (5) and mouthpiece (10) are pulled apart, the capsule is inserted and the two parts of the inhaler are fitted together. After being pressed back into position (36) counter to the spring element (35) the cutting device (19) is actuated and released again. Under the pressure of the spring element (35) the inhaler returns to the initial position shown in FIG. 4. The active substance formulation from the capsule (not shown) can now be inhaled by breathing in through the mouthpiece (10).

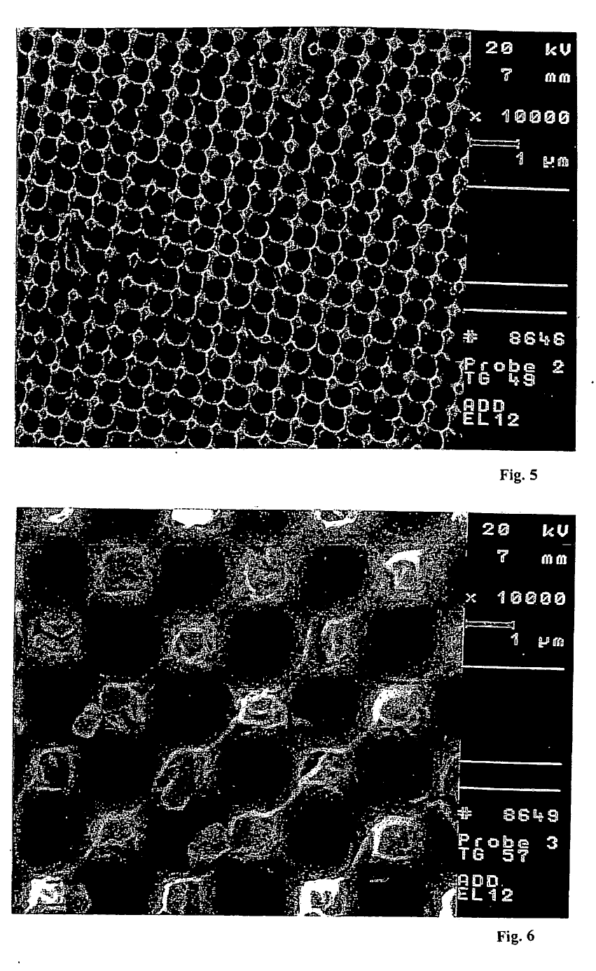

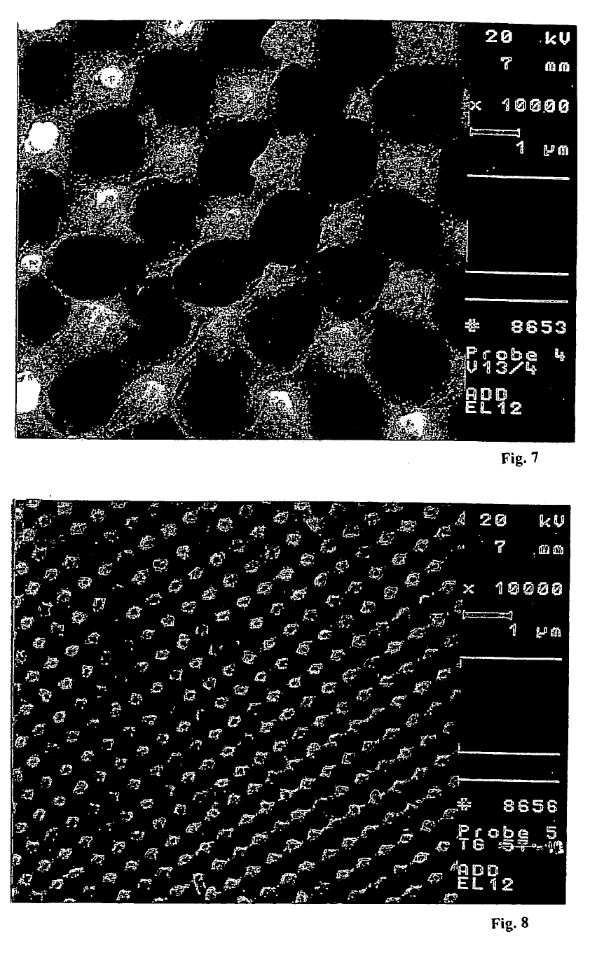

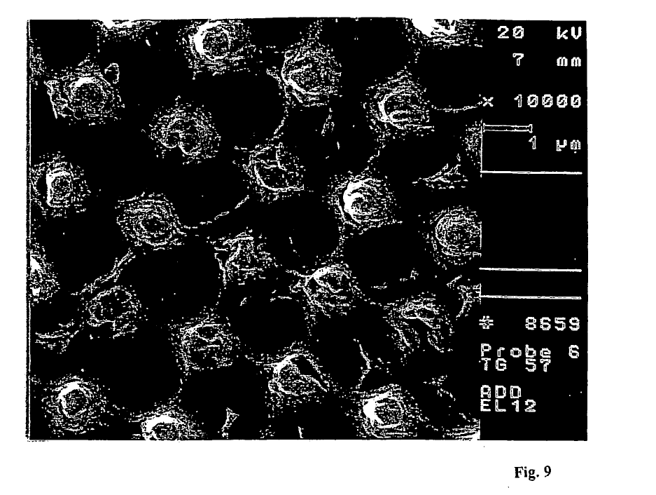

FIGS. 5 to 9 show examples of surface structures, specifically the surface structures of the films according to Example 3.

Film 1 with structures in the region of 0.5 microns,

Film 2 with structures in the region of 2 microns,

Film 3 with structures in the region of 2 microns and 10 microns of superstructure.

1. An inhaler for the administration of a pharmaceutical composition comprising a mouthpiece, an air channel opening into the mouthpiece and optionally a chamber which may optionally be provided with an air inlet channel or an air opening, wherein the inhaler is capable of receiving a) a capsule with the composition, b) a blister pack with the composition, or c) a conveyor belt holding the composition, wherein at least part of the inner surface of the mouthpiece and/or of the air channel and/or optionally the chamber contains elevations and/or depressions with a height/depth of from 0.1 to 100 microns.

2. The inhaler according to claim 1 wherein at least either the inner surface of the mouthpiece, the air channel and/or the chamber is produced by microtechnology or nanotechnology over at least 20% of its surface.

3. The inhaler according to claim 1 wherein the elevations and depressions are separated by spacings in the range from 0.1 to 200 microns.

4. The inhaler according to claim 1 wherein the inner surfaces are formed by hydrophobic materials selected from one or more of glass, ceramics, metals and plastics, wherein the plastics are further selected from one or more of polyethylene, polypropylene, polycarbonate, polyacrylate, polyester and silanes.

5. The inhaler according to claim 1 wherein the inner surfaces are formed by processes comprising subtractive or additive treatment selected from stamping, etching, laser ablation, galvanic machining, adhesively attaching a structured film, adhesion of a powder, spraying with suspensions, and depositing sublimates.

6. The inhaler according to claim 1 wherein the inhaler is a Bernoulli inhaler.

7. The inhaler according to claim 6 wherein the inhaler comprises a capsule chamber which is connected to the air channel opening in the mouthpiece.

8. The inhaler according to claim 7 wherein the capsule chamber has a diameter 1.1 to 2.5 times the capsule diameter and a length 1.02 to 2 times the length of the capsule.

9. The inhaler according to claim 7 wherein the inhaler has a cutting device which is fitted with at least two sharp spikes and/or cutters, the spikes and/or cutters being capable of being inserted through openings into the capsule chamber(s).

10. The inhaler according to claim 6 wherein the inhaler comprises
a) a cup-shaped lower part open at the top,
b) a plate which covers the opening of the lower part and perpendicularly to which is formed the capsule chamber, a button movable counter to a spring on the capsule chamber, comprising two sharp spikes or cutters for opening the capsule,
c) an upper part with the mouthpiece and the air channel which connects the mouthpiece to the capsule chamber so as to be able to convey a powder or liquid or aerosol, and
d) a lid, the elements a), b) c) and d) being joined together by a common hinge element such that they can be moved back and forth relative to one another.

11. A capsule for use with an inhaler wherein the inner and/or outer surface of the capsule is structured by elevations and/or depressions with a height/depth of 0.1 to 100 microns.

12. A blister pack adapted for use with an inhaler wherein the inner and/or outer surface of the capsule retained in each blister comprises elevations and/or depressions with a height/depth of 0.1 to 100 microns.

13. A membrane for conveying a metered amount of active substance for an inhaler wherein the inner and/or outer surface of the membrane comprises elevations and/or depressions with a height/depth of 0.1 to 100 microns.

14. The blister pack according to claim 12 wherein the inner surface and/or outer surface of the capsule is produced by microtechnology or nanotechnology.

15. The capsule according claim 11 wherein the inner surface and/or outer surface is produced by microtechnology or nanotechnology.

16. The membrane according to claim 13 wherein the inner surface and/or outer surface is produced by microtechnology or nanotechnology.

17. The blister pack according to claim 14 wherein the surface of the capsule is produced by microtechnology or nanotechnology over at least 20% of its surface.

18. The blister pack according to claim 17 wherein the spacings between the elevations and depressions of the surface of the capsule are in the range from 0.1 to 200 microns.

19. The capsule according to claim 15 wherein the surface is produced by microtechnology or nanotechnology over at least 20% of its surface.

20. The capsule according to claim 19 wherein the spacings between the elevations and depressions of the surface are in the range of from 0.1 to 200 microns.

21. The membrane according to claim 16 wherein the surface is produced by microtechnology or nanotechnology over at least 20% of its surface.

22. The membrane according to claim 21 wherein the spacings between the elevations and depressions on the surface are in the range of from 0.1 to 200 microns.
